(19) United States
(12) Patent Application Publication
(10) Pub. No.: US 2022/0001071 A1
(43) Pub. Date: Jan. 6, 2022

(54) SANITIZING SYSTEMS AND METHODS FOR COMMERCIAL PASSENGER VEHICLES

(71) Applicant: Bruce Aerospace Inc., Dayton, NV (US)

(72) Inventors: Adam W. Brush, Reno, NV (US); Storm S. Sears, Dayton, NV (US)

(21) Appl. No.: 17/365,497

(22) Filed: Jul. 1, 2021

Related U.S. Application Data
(60) Provisional application No. 63/046,898, filed on Jul. 1, 2020.
(60) Provisional application No. 63/075,913, filed on Sep. 9, 2020.

(51) Int. Cl. A61L 9/20; A61L 2/10; B64D 13/08

(52) U.S. Cl. CPC A61L 9/205; A61L 2/10; B64D 13/08; A61L 2209/111; B64D 2013/003; A61L 2209/16; A61L 2202/11; A61L 2202/25; A61L 2209/12

(57) ABSTRACT

The invention relates to a sanitation treatment system and methods for use in association with commercial passenger vehicles such as aircraft. The sanitation system may operate along with a filtration system in an air distribution system associated with an aircraft or like commercial passenger vehicles and/or to provide disinfected air and surfaces in the passenger cabin associated with the aircraft or like vehicle.

### CROSS REFERENCE TO RELATED APPLICATIONS

This application claims benefit of pending U.S. Provisional Patent Application Nos. 63/046,898, filed Jul. 1, 2020 and 63/075,913 filed Sep. 9, 2020, the contents of which are incorporated herein by reference.

### TECHNICAL FIELD

The invention relates to sanitizing systems and methods for use in association with a filtration system in an air handling system associated with an aircraft or like commercial passenger vehicles and/or to provide disinfected air and surfaces in the passenger cabin associated with the aircraft or like commercial passenger vehicles.

### BACKGROUND OF THE INVENTION

The spread of disease in commercial passenger vehicles such as an aircraft, trains or the like, has been an issue for some time, and has become more prevalent with increased use of such commercial passenger vehicles and new dangerous pathogens being encountered in the environment. Though aircraft and like vehicles have air handling systems that may include a filtering system to attempt to remove contaminants from the air being distributed to the passenger cabin, the possibility of transmission of pathogens such as microorganisms (e.g., various bacteria), and viruses in the air is still present. There is therefore a significant continued need to effectively remove pathogens from the air distributed to the passenger cabin of aircraft and like vehicles. There has been the use of High Efficiency Particulate Air (HEPA) filters to attempt to remove contaminants from the air, they may not remove gaseous or molecular agents such as some bacteria or viruses. The HEPA filters are used for particulate removal, but are only 99.97% efficient at collecting the most penetrating particle (−0.2 micrometer). Though effective, HEPA filters may be vulnerable to viruses that are submicron in size, and which have very small minimum infective dose (MID). Therefore, an appropriate viral challenge will yield penetration that exceeds the MID, for many viruses. Though aerosolized viruses are commonly thought to exist as agglomerates, which would increase the particle size and render them more prone to capture, even agglomerated virus particles still exist as submicron particles, or may break apart during filtration. There is therefore a continued need for an effective system and method to disinfect air distributed to the passenger cabin of the aircraft or like commercial passenger vehicle, particularly destroying viruses, bacteria or other pathogens.

The capabilities and benefits of UV light to kill and destroy bacteria or viruses in the air is known, but UV light is also dangerous to humans. Though broad-spectrum germicidal UV light, with wavelengths between 200 and 280 nanometers (nm), is highly effective at killing bacteria and viruses by destroying the molecular bonds that hold their DNA together, use in an environment where people are is generally not acceptable.

In the case of commercial passenger vehicles, such as an aircraft, such objectives of a disinfecting system must be accomplished by a system within the constraints of weight and size of the air distribution system of the commercial passenger vehicle, as well as pressure drop across the system in the case of an aircraft. There is therefore a need for a system and method that allows better cleansing of the air distributed to the passenger cabin of a commercial passenger vehicle.

There is a further need for an air distribution system for an aircraft that not only removes particulates, but also inactivates viruses, bacteria, and other microorganisms, and works to improve the performance of a HEPA or like filter used to remove contaminants in air handling systems of aircraft or other commercial passenger vehicles. It would also be highly desirable to allow for disinfection of air and/or surfaces in the passenger seating areas of aircraft or the like.

### SUMMARY OF THE INVENTION

In one aspect of the present invention, there is provided at least one UV LED sanitizing array that operates with a HEPA filter assembly in commercial passenger vehicles such as aircraft, as a part of the cabin air recirculation systems, for the purpose of enhancing the performance of the air filtration system. The at least one UV LED sanitizing array will irradiate the surfaces of the HEPA filter with UV radiation in the anti-germicidal wavelength range, inactivating viral microbes in the air adjacent the filter and that have become entrapped by the filter.

The invention is also directed to a supplemental UV irradiation system for incorporation into use with the filter used in an air distribution system of an aircraft or the like. The supplemental system is positionable in relation to the filter system to disinfect the surfaces of the filter system.

The invention also relates to a method of treating the air distributed to the passenger cabin of a commercial passenger vehicles, by causing flow of air through an air handling system and through at least one HEPA filter. At least one UV LED sanitizing array is positioned adjacent the at least one HEPA filter assembly and operated to irradiate the at least one surface of the at least one HEPA filter to enhance the performance of the air filtration system.

In another aspect, the invention relates to an irradiation system configured to irradiate the air recirculating to and from the cabin of a commercial passenger vehicle, and/or irradiation of at least a portion of the interior of the cabin with far-UV radiation and method. The irradiation system may be associated with the air distribution outlets in the passenger cabin and/or adjacent exposed surfaces in the passenger areas.

These and other aspects of the invention will become apparent based upon the following description of examples of the invention in conjunction with the figures.

### BRIEF DESCRIPTION OF THE DRAWINGS

FIG. 1 is a somewhat schematic view of a first example of the invention showing an air treatment system including at least one filter and an irradiation system to treat at least one surface of the filter in the treatment system;

FIG. 2 shows an enlarged section of the system of FIG. 1, according to the example of the invention;

FIG. 3 shows an example of an irradiation emitter arrangement according to an example;

FIG. 4 shows an alternate example of the invention with an example irradiation system for incorporation into association with at least one filter in the air distribution system of an aircraft;

FIG. 5 shows an enlarged section of the system of FIG. 4, according to the example of the invention;

FIG. 6 schematically represents a filter in an air distribution system for an aircraft, and a supplemental irradiation system according to another example of the invention in association therewith;

FIG. 7 shows an enlarged portion of the example of FIG. 6;

FIG. 8 schematically represents a filter in an air distribution system for an aircraft, and a supplemental irradiation system according to another example of the invention in association therewith;

FIG. 9 schematically represents a filter in an air distribution system for an aircraft, and a supplemental irradiation system according to another example of the invention in association therewith;

FIG. 10 shows a somewhat schematic view of another example of the invention showing a treatment system including at least one irradiation system to treat the circulating air and/or at least one surface of the interior of the passenger cabin or like interior compartment of the aircraft;

FIG. 11 shows a flow diagram of a method according to an example of the invention;

FIG. 12 shows a flow diagram of a method according to an example of the invention; and

FIG. 13 shows another example of a sanitizing system according to the invention.

### DETAILED DESCRIPTION OF THE INVENTION

The present invention provides an enhanced disinfecting treatment system and methods for commercial passenger vehicles such as aircraft. The systems and methods enable more effective removal of contaminates including viruses, bacteria or other microorganisms from recirculated air to and from the passenger cabin or surfaces and in other areas where passengers or flight personnel are located. As an example, the present invention may be used to provide enhanced air treatment and/or disinfection of the interior air space(s) of a commercial aircraft. A method of the invention may also be applicable to such commercial passenger vehicles, to treat the recirculated air stream in conjunction with a filter, to be delivered to the interior air space of the vehicle, and/or to treat at least a portion of the interior surface of the cabin or like location in the aircraft or other vehicle.

Air provided to an interior cabin in an aircraft or the like may be contaminated by passengers in the cabin or from other sources, and it is desired to remove pollutants or microbe from the air. It has been attempted to use a HEPA filter in the air distribution system, but while HEPA filters entrap particles as small as 0.3 microns in size, with an effectiveness as high as 99.97%, such filters may only be marginally effective on some virus, bacteria or other microbe particles, which can range in size between 0.075 to 0.2 microns in diameter for example. In addition, an accumulation of viral particles within the filter over time may allow these particulates to work their way through the filter. The present system and method inactivate the viral or other pathogen particles as they approach and embed themselves on the filter surface, and the long-term effectiveness of the filter system can be assured. The system and method may be used to augment the HEPA filter with an anti-germicidal UVC array that can increase the filtering effectiveness of the HEPA filter against viral particulates by 1 Log, to a level of 99.999% effectiveness.

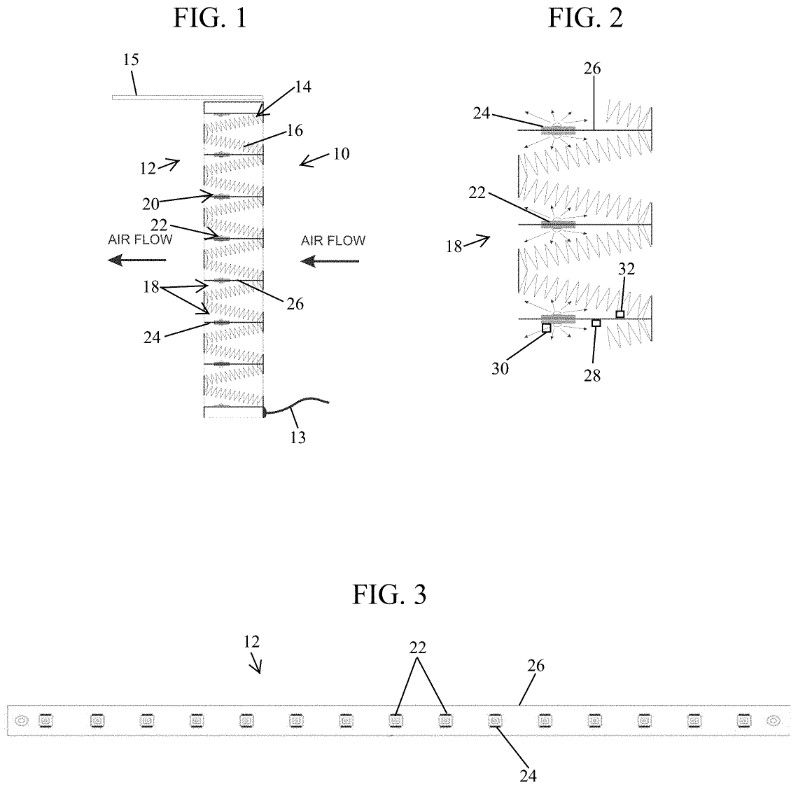

In a first example of the invention as shown in FIG. 1, a treatment system 10, includes a UVC LED array 12 provided in association with at least one filter 14. The filter 14 may be a HEPA filter, or other filter which effectively removes contaminants from the air as the air passes through filter 14 in an air distribution system of an aircraft. The combination of the irradiation system 12 and filter 14 is formed as a particular configuration with a form factor to match the filter housing (not shown) in the air distribution system of the aircraft if present, or designed to correspond to a housing or duct associated with the air distribution system in original equipment designs. The system 10 may be mounted in an existing duct system 15 of an aircraft air distribution system for example. The HEPA filter 14 may itself be of various configurations and form factors for a particular system 10, but in all cases will include a filter media 16 extending over the region of a duct 15, plenum or other housing in the air distribution system. The filter media 16 is many times pleated or the like to increase the surface area of the filter media 16. In this example, the pleats 18 are formed over the surface of filter media 16, on at least one side, but possibly both sides to allow more effective filtration. The irradiation system 12 is then formed to include a plurality of irradiation elements 20, that include at least one UV radiation source 22, such as an LED bulb, or possibly a fluorescent bulb, excimer lamp or other suitable source, that emits UV germicical radiation (UVG) including UVC and/or far-UV radiation onto the surfaces of the pleat 18. As different pathogens react somewhat differently to UVC and far-UV radiation, the array may also include germicidal radiation sources 22 of different wavelengths that target certain pathogens for example. In the example shown, UVG LED's 22 are positioned on the first and second sides of a printed circuit board (PCB) board 24 on which the LED's 22 are mounted. The irradiation of the surfaces of the filter 14 enable effective killing of virus particles entrapped in the filter and as the air passing through the filter 14 is slowed by the filter itself, the air adjacent the filter is also effectively irradiated to kill airborne pathogens. The system 10 may also include UVC and/or far-UV sources 22 at a spaced position from the filter 14, to treat air in the ductwork of the air distribution system downstream and separate of the HEPA filter 14, irradiating either the filter 14 surfaces at a distance and/or the air column directly.

The array 12 is positioned in close proximity to the pleats 18 of filter 14 as seen in FIG. 2, and provide enhanced disinfection, as the UV radiation is better capable of penetrating the fibrous pleats 18 to some degree and treating all air passing through the filter 14. As microbes must have direct exposure to the UV radiation in order for it to kill them, this placement of the array 12 effectively treats the air as well as surfaces of the filter 14. This placement also provides UV radiation in the dosage, distance, intensity, and duration of exposure to more effectively kill pathogens in conjunction with the filter 14. Generally, the UV dosage amounts decrease significantly by approximately the square of the distance from the source light, so the placement directly adjacent the surfaces of filter 14 avoids attenuation of the UV dosage. The intensity of the UV radiation sources 22 and distance from the surfaces of filter media 16 are chosen to provide the desired efficacy in killing the pathogens. The array 12 may position the UV radiation sources 22 within 2 cm of the surfaces of filter media 16, to reduce the energy required from the UV radiation sources 22 for example, though other configurations are contemplated. The UV intensity amount required to kill most bacteria and viruses is 2,000 to 8,000 [tuts/cm²], and the array 14 may provide the desired dosage.

As seen in FIG. 3, the UV LED boards 24 in this example, may be mounted to supports 26. In this example, the array 12 is linear to extend the entire length of the pleat 18 in which it is positioned, but the array 12 may be of any desired configuration. The individual supports 26 may be connected together or mounted directly with the filter 14. The supports 26 may be formed of aluminum which reflects the UV radiation, but because of the full and direct irradiation of all surfaces of filter 14, the supports do not have to be reflective. The boards 24 themselves may provide the desired structure to mount the arrays 12, and no supports 26 may be required. The supports 26 may also provide heat-sinking for the light sources 22 and boards 24 to keep the UV emitters at an optimal operating temperature. The geometry of the supports 26 and LED PCB assemblies do not to impede air flow through the HEPA filter and also to create minimal turbulence. The LED boards 24 are oriented generally perpendicularly to the filter 14, but being positioned in the pleats 18 of filter 14 allow all surfaces to be equally irradiated by the UV radiation as seen in FIG. 2. The LED's 22 may be coated to only emit UV radiation that is outside of the ozone producing wavelengths to ensure its safe utilization in an indoor space around occupants or service personnel. Further, the LED's 22 may be configured to emit UVC or far-UV radiation in specific wavelengths for optimized performance in the environment of a commercial aircraft or the like. For example, the LED's 22 may be configured to emit UV in the energy range of about 3.5-10 eV to provide high-energy photons to cleave most chemical bonds and kill microbes by destroying nucleic acids and disrupting their DNA.

In this example, the UV LED emitters 22 are surface mounted, and emit energy in the optimal anti-germicidal band, such as between 200 to 280 nm for example. The UV LED emitters 22 and boards 24 are the same width or length of the pleats 18 in the HEPA filter 14 to be disinfected. An example of such a LED board assembly 24 is depicted in FIG. 3. The LED board assemblies 24 also incorporate the current regulation circuitry for the UV emitters 22 to keep them operating at a precise operating current level over temperature. The LED board assemblies may incorporate a lens assembly formed from special UV transmissive silicone rubber, in order to protect the UV LED emitters 22 from physical damage.

The UVC sanitizing array assembly 12 may be powered from available aircraft 28 Vdc or 115 Vac, 400 Hz power. The UV array assembly 12 can be directly powered from aircraft 28 Vdc power, via power connection 13. In the case of 115 Vac, 400 Hz power distribution, a separate power supply unit (not shown), mounted separately from the UV array 12, will be provided to convert 115 Vac power to 28 Vdc. The UV sanitizing array 12 electronics assembly may also incorporate an air motion sensor 28 to turn off the UV array 12 in the event that the cabin air system is inactivated while power is still supplied to the UV array 12. There may also be provided a temperature monitor 30 to detect any elevated temperature of the LED's or other sources and provide a warning or temporarily suspend operation of the sources 22. There may also be provided a UV sensor 32 to detect the amount of UV being emitted an indicate if insufficient UV irradiation or possible exposure to personnel is occurring.

The HEPA filtration system 14 is generally located in unmanned areas of the aircraft during flight and thus, any minor amount of UVC radiation that may be harmful to humans escaping from the UVC LED array 12, particularly when the irradiation system 12 is employed on the intake side of the HEPA filter system 14, poses no hazard to flight crew or passengers. Nonetheless, appropriate safeguards may be taken to disable the apparatus and prevent potential exposure to maintenance personnel while the aircraft is on the ground. Such safeguards may take the form of secondary radiation shields, electrical power interlock mechanisms, proximity sensors or other suitable devices to alert personnel of the system 10. In this example, as the UVC LED array 14 is positioned in close proximity to the HEPA filter 14 surfaces for maximum effectiveness, the radiation will not generally extend beyond the location of filter 14 to any significant extent, and so that the lowest feasible amount of UVC radiation can be employed to lower the power consumption of the unit for a cost effective system 10. The radiated UVC power level and potential for damage to skin or eyes with prolonged UVC exposure falls off radically with just a few feet of physical separation from the apparatus 10, and momentary exposure poses no health risk.

The system 10 that incorporates the UV LED array 12 directly into the HEPA filter 14 construction provides the advantage in allowing a drop-in replacement for an existing filter in an aircraft, requiring only a simple wired connection to 28 Vdc supply power. The irradiation array 12 may be provided on the exhaust side of the filter 14, which is the side facing the air system ductwork, to eliminate any concern of stray UVC radiation exposure for maintenance personnel. The UV LED emitters 22 are positioned in a manner that there is no need for a reflector system to capture and direct stray radiation, increasing radiative efficiency. The UV array 12 is positioned very close to the surfaces of the filter material, requiring less emissive power to obtain the desired germicidal effect. Thus, lower power, lower cost UV LED emitters 22 can be employed in the design and less input power is required to run the UV LED array 12. The use of low-cost, lower-power UVC or far-UV emitters 22 makes it feasible for the UV array 12 to be disposable along with the HEPA filter 14 after a period of time. This ensures that there is no degradation over time of the system 10 or its components either due to natural decay of LED emissive power over time, or by degradation of materials or contamination of the UV emitter lens or other components.

The UV emitters 22 may be configured to emit far-UV in the range of 200 to 222 nanometers, which effectively eliminates pathogens, but does not adversely affect occupants or service personnel that may be exposed to it. This shorter wavelength renders it unable to pass through the barrier of non-living cells on skin, and tears on the surface of the eye. At the same time, that wavelength is what makes it very effective in penetrating and inactivating bacteria and viruses. The light sources 22 may include an optical bandpass filter to emit the desired wavelength of UV radiation onto the filter 14, or the light sources 22 could be monochromatic or quasimonochromatic to emit UV of a desired wavelength or small range of wavelengths. It should be noted that this is only an example of the system 10 for a configuration of HEPA filter 14 and irradiation system 12 associated therewith, and the shapes and configurations of the filter 14 and associated irradiation system can be modified to correspond to an existing filter housing associated with an air distribution system of an aircraft or like commercial passenger vehicle, or be configured as original equipment in the distribution system. In any event, the system 10 is positionable in the path of the air flow to cleanse the air as well as disinfecting it along with the surfaces of the filter 14. The system 10 is then removable and replaceable to periodically provide a fresh filter 14.

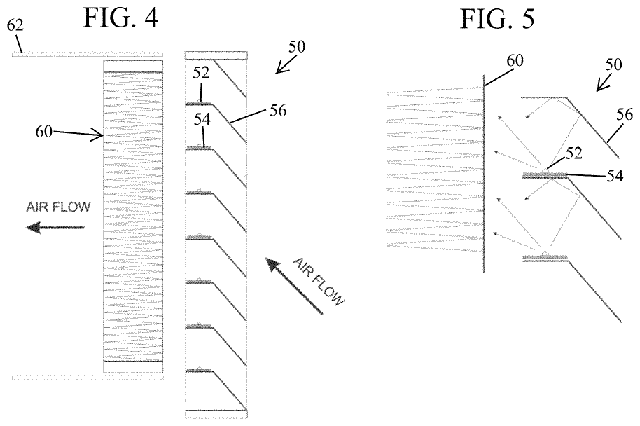

Turning to FIGS. 4 and 5, another example of the invention relates to a supplementary UVC irradiation system 50 that is incorporated for use with an existing filter 60 of an air distribution system of an aircraft or the like. The filter 60 is mounted in a duct 62 of the air distribution system to filter air being recirculated to the passenger cabin or other locations in the aircraft. The irradiation system 50 includes a plurality of UVC and/or far-UV emitters 52 that are arrayed on Printed Circuit Board (PCB) assemblies 54 that are supported on a support 56. The irradiation system 50 and support 56 are configured to correspond to the shape and configuration of the HEPA filter 60 to be disinfected. The system 50 may use linear assemblies 54 similar to that shown with respect to the example of FIG. 3, or any configuration to provide irradiation of the entire surfaces of filter 60. The LED board assemblies 54 also may incorporate the necessary current regulation circuitry for the UV emitters to keep them operating at a precise operating current level over temperature. The LED board assemblies as necessary, and again may incorporate a lens assembly formed from special UV transmissive silicone rubber, in order to protect the UV LED emitters 52 from physical damage. The UV sanitizing array assembly 50 may again be powered from available aircraft 28 Vdc or 115 Vac, 400 Hz power as may be desired. The UV sanitizing array 50 may also incorporate air motion sensing circuitry to turn off the UV array 52 in the event that the cabin air system is inactivated while power is still supplied to the UV array 52, or other sensors or attributes as described with reference to the example of FIG. 1.

The UV sanitation assemblies 50 are mounted and located in close proximity to the surface of the HEPA filter 60 using an open aluminum, louvered framework 56. The aluminum framework 56 affords support of the UV LED PCB assemblies 54, as well as, providing heat-sinking for the LED boards 54 to keep the UV emitters 52 at an optimal operating temperature. The UV array 50 can be located near the intake or exhaust surface of the HEPA filter, where it will work with equal effectiveness.

The geometry of the supporting framework 56 is designed so as not to impede air flow through the HEPA filter 60 and also to create minimal turbulence. This means that in most cases, the LED boards 54 will be oriented perpendicular to the surface of the filter 60, which makes it desirable to reflect a portion of the radiated energy emitted by the UV LEDs back towards the filter 60, so as not to waste this energy. This can be accomplished in various suitable ways. In this example, the louvered aluminum support framework 56 for the UV LED boards 54 is depicted. The aluminum louvers 56 serve to both support the LED boards 54 and to also reflect a portion of the UV radiation emitted from the LED array 52 that would otherwise be lost, back towards the HEPA filter surface by means of angled louvers, as shown in FIG. 5. The LED UV emitters 52 may also be side-emitting LED's for direct irradiation of the filter 60 surfaces. Aluminum surfaces are highly reflective to UV wavelengths, and the UV LED boards 54 and UV emitters 52 are spaced in a manner such that uniform UV irradiation of the HEPA filter 60 surfaces is achieved. If desired, the orientation of the reflecting portions of supports 56 may be fixed or variable to minimize interference with airflow.

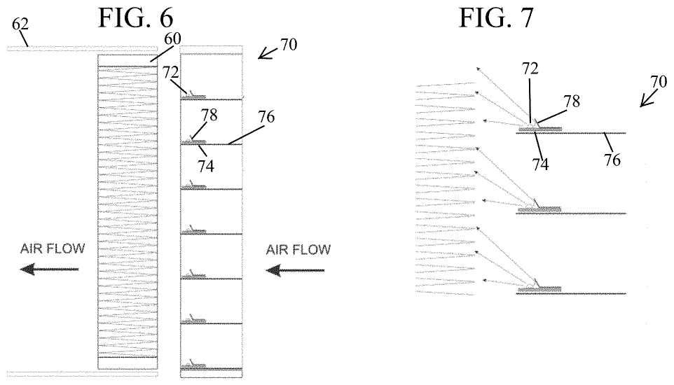

Another example of the invention is shown in FIGS. 6 and 7, showing an alternative supplemental UV irradiation system 70. In this arrangement, the system 70 is again incorporated for use with an existing filter 60 of an air distribution system of an aircraft or the like. The filter 60 is mounted in a duct 62 of the air distribution system to filter air being recirculated to the passenger cabin or other locations in the aircraft. The irradiation system 70 includes a plurality of UV LED emitters 72 that are arrayed on PCB assemblies 74 that are supported on a support 76. The irradiation system 70 and support 76 are configured to correspond to the shape and configuration of the HEPA filter 60 to be disinfected. The system 70 may use linear UV emitter assemblies 74 similar to that shown with respect to the example of FIG. 3, or any configuration to provide irradiation of the entire surfaces of filter 60. The LED board assemblies 74 also may incorporate the necessary current regulation circuitry for the UV emitters 72 to keep them operating at a precise operating current level over temperature. The UV sanitizing array assembly 70 may again be powered from available aircraft 28 Vdc or 115 Vac, 400 Hz power as may be desired. The UV sanitizing array 70 may also incorporate air motion sensing circuitry to turn off the UV array in the event that the cabin air system is inactivated while power is still supplied to the UV array, or other sensors or attributes as described with reference to the example of FIG. 1.

The UV sanitation assemblies 70 are mounted and located in close proximity to the surface of the HEPA filter 60 using an open aluminum, vaned framework 76. The aluminum framework 76 affords support of the UV LED PCB assemblies 74, as well as, providing heat-sinking for the LED boards 74 to keep the UV emitters 72 at an optimal operating temperature. The UV array 70 can be located near the intake or exhaust surface of the HEPA filter 60, where it will work with equal effectiveness.

The geometry of the supporting framework 76 is designed so as not to impede air flow through the HEPA filter 60 and also to create minimal turbulence. To facilitate this, the LED boards 74 are oriented perpendicular to the surface of the filter 60, which makes it desirable to reflect a portion of the radiated energy emitted by the UV LEDs back towards the filter 60, so as not to waste this energy. In this, example, this is accomplished by reflection of UV toward the filter 60 by small, low-profile aluminum reflectors 78 that are incorporated directly upon the UV LED boards 74 themselves. The height of the reflector 78 only needs to be just above the top surface of the UV LED emitters 72 and thus, the reflectors 78 will not significantly impede airflow into the HEPA filter 60. The LED UV emitters 72 may also be side-emitting LED's for direct irradiation of the filter 60 surfaces. Aluminum surfaces are highly reflective to UV wavelengths, and the UV LED boards 74 and UV emitters 72 with reflectors 78 are spaced in a manner such that uniform UV irradiation of the HEPA filter 60 surfaces is achieved.

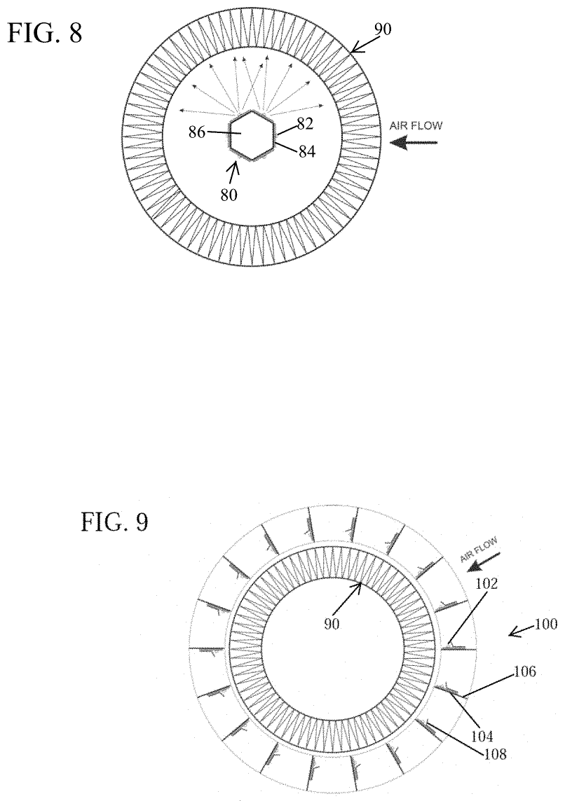

Another example of the invention is shown in FIG. 8, showing an alternative supplemental UV irradiation system 80. In this arrangement, the system 80 is again incorporated for use with an existing filter 90 of an air distribution system of an aircraft or the like. In this example, the filter 90 is cylindrical and is mounted in a duct of the air distribution system to filter air being recirculated to the passenger cabin or other locations in the aircraft. The irradiation system 80 includes a plurality of UV LED emitters 82 that are arrayed on PCB assemblies 84 that are supported on a support 86. The irradiation system 80 and support 86 are configured to irradiate the cylindrical surfaces of the HEPA filter 90 to be disinfected. The system 80 may use linear assemblies 84 similar to that shown with respect to the example of FIG. 3, or any configuration to provide irradiation of the entire surfaces of filter 90. Again, the LED board assemblies 84 also may incorporate the necessary current regulation circuitry for the UV emitters to keep them operating at a precise operating current level over temperature. The UV sanitizing array assembly 80 may again be powered from available aircraft 28 Vdc or 115 Vac, 400 Hz power as may be desired. The UV sanitizing array 80 may also incorporate air motion sensing circuitry to turn off the UV array in the event that the cabin air system is inactivated while power is still supplied to the UV array, or other sensors or attributes as described with reference to the example of FIG. 1.

The UV sanitation assemblies 80 are mounted and located in close proximity to the interior cylindrical surfaces of the HEPA filter 90 using a multi-sided support 86. The support may be dimensioned to position the UVC emitters 82 a desired distance from the filter 90 surfaces. The support 86 may also provide heat-sinking for the LED boards 84 to keep the UV emitters 82 at an optimal operating temperature. The geometry of the system 80 is designed so as not to impede air flow through the HEPA filter 90.

Another example of the invention is shown in FIG. 9, showing an alternative supplemental UV irradiation system 100. In this arrangement, the system 100 is again incorporated for use with an existing filter 90 of an air distribution system of an aircraft or the like. In this example, the filter 90 is again cylindrical and is mounted in a duct of the air distribution system to filter air being recirculated to the passenger cabin or other locations in the aircraft. The irradiation system 100 includes a plurality of UV LED emitters 102 that are arrayed on PCB assemblies 104 that are supported on a support 106. The irradiation system 100 and support 106 are configured to irradiate the outer cylindrical surfaces of the HEPA filter 90 to be disinfected. The system 100 may use linear assemblies 104 similar to that shown with respect to the example of FIG. 3, or any configuration to provide irradiation of the entire surfaces of filter 90. Again, the LED board assemblies 104 also may incorporate the necessary current regulation circuitry for the UV emitters to keep them operating at a precise operating current level over temperature. The UV sanitizing array assembly 100 may again be powered from available aircraft 28 Vdc or 115 Vac, 400 Hz power as may be desired. The UV sanitizing array 100 may also incorporate air motion sensing circuitry to turn off the UV array in the event that the cabin air system is inactivated while power is still supplied to the UV array, or other sensors or attributes as described with reference to the example of FIG. 1.

The UV sanitation assembly 100 is mounted and located in close proximity to the outer surface of the HEPA filter 90 using an open aluminum, vaned framework 106. The aluminum framework 710 affords support of the UV LED PCB assemblies 104, as well as, providing heat-sinking for the LED boards 710 to keep the UV emitters 102 at an optimal operating temperature. The UV array 100 can be located near the intake surface of the HEPA filter 90.

The geometry of the system 100 and supporting framework 106 is designed so as not to impede air flow through the HEPA filter 90 and also to create minimal turbulence. To facilitate this, the LED boards 104 are oriented perpendicular to the surface of the filter 90, and reflection of UV toward the filter 90 may again be provided by small, low-profile aluminum reflectors 108 that are incorporated directly upon the UV LED boards 104 themselves. The height of the reflector 108 only needs to be just above the top surface of the UV LED emitters 102 and thus will not significantly impede airflow into the HEPA filter 90. The LED UV emitters 102 may also be side-emitting LED's for direct irradiation of the filter 60 surfaces. Aluminum surfaces are highly reflective to UV wavelengths, and the UV LED boards 74 and UV emitters 72 with reflectors 78 are spaced in a manner such that uniform UV irradiation of the HEPA filter 60 surfaces is achieved.

In the examples above, it may also be desirable to provide a pre-filter (not shown) in association with the irradiation system, to filter larger particulates that may be in the circulated air. This may extend the life of the HEPA or other like filtration system. It also may be desirable to cause the speed of the circulated air to be slowed for treatment with the irradiation system of the invention, and dampers, baffles or any suitable arrangement to provide such a function can be used. Also, the velocity of the air could be diminished by a housing, partial barrier, opposing fan, or the like. The examples of the invention are also configured to operate at the expected temperature and pressure environments experienced on an aircraft, while performing in the desired manner.

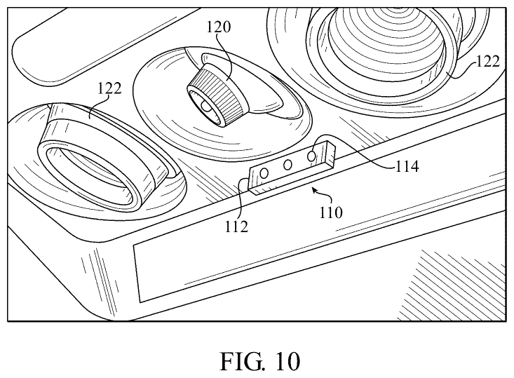

Another example of the invention is shown in FIG. 10, showing an alternative supplemental UV irradiation system 110. In this arrangement, the system 110 is provided for disinfection of recirculating air in the cabin of the aircraft, as well as disinfection of high touch surface areas in the passenger area for example. In this example, the system 110 includes a housing 112 with at least one UV irradiation source 114. The UV irradiation source 114 is a far-UV emitter, providing radiation with a wavelength of between about 200 and 222 nm. Such radiation provides disinfection of the air and surfaces in close proximity to the at least one source 114. Positioning the housing 112 adjacent the air blower 120 provided for passengers of the aircraft will irradiate the recirculated air before impinging upon the passenger. The adjacent surfaces also include personal reading lights 122, and the entire area is a high touch area of the passenger seating area. The at least one UV irradiation source 114 may be configured to directly irradiate these high touch surfaces with the far-UV radiation to disinfect the surfaces continuously. It should be apparent that other high touch areas of the passenger compartment or the like may be treated using a suitable housing and at least one UV irradiation source 114. To facilitate treatment of the recirculating air in the cabin, there may be provided a chamber to allow for additional treatment exposure to the radiation emitted from the at least one source 114. Such a chamber or other suitable structures may be used to cause the speed of the circulated air to be slowed for treatment with the irradiation system of the invention. Alternatively or in addition, other structures may be used to slow the air for treatment, such as dampers, baffles or the like. As an example, the at least one UV irradiation source 114 may emit UV at a wavelength of about 200 to 222 nm, which is efficient at low dose rates to kill pathogens in seconds. Such UV radiation has a higher absorption rate in pathogens such as bacteria and viruses, and is also safer for use around humans because it cannot penetrate skin or eyes. This radiation physically destroys pathogens due to its shorter wavelength and higher photon energy, which chemically changes bonds and causes mutations that prevent cells from replicating, but does not destroy them. The provision of discrete systems 110 to enable effective disinfection of air and surfaces in the passenger cabin provides distinct advantage in controlling the possible spread of disease. It should also be recognized that the form factor of housing 112 may be of any suitable configuration to provided disinfecting irradiation where it is desired. The system 110 may be integrated into the air blower, reading lights The far-UV source 114 may be a suitable source such as LED, excimer lamp or the like. The desired wavelength(s) around 200-222 nm may be provided by providing the source as a monochromatic or quasimonochromatic source to emit far-UV of a desired wavelength or small range of wavelengths. For example, far-UVC light generated by filtered LED's or excimer lamps emitting in the 207 to 222 nm wavelength range, can be used to efficiently inactivate pathogens. The irradiation sources 114 may alternatively or in addition include an optical bandpass filter to emit the desired wavelength of far-UV radiation to treat the air and/or onto the adjacent surfaces for disinfection. The systems 110 can be used continuously over an extended period of time, to facilitate prevention of disease transmission. Other sanitation systems according to this example may be provided in the aircraft cabin, such as a ceiling mounted treatment system to draw cabin air and treat it continuously. A separated circulation system could be provided to draw cabin air to the system for treatment. A separate housing or chamber may be provided to allow treatment of the air near the ceiling of the cabin. A treatment system may also be incorporated into the passenger service units (PSU) or air gaspers in the cabin.

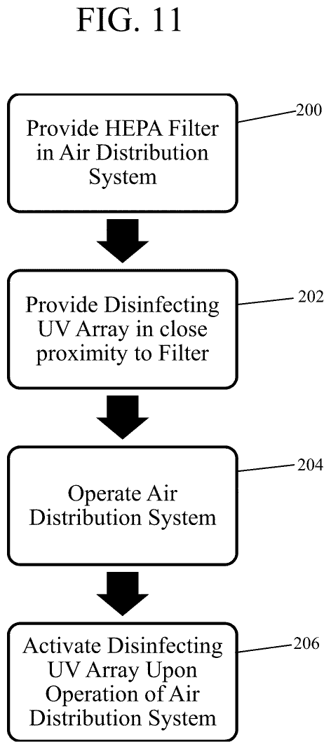

FIG. 11 schematically represents a series of steps involved in a method for removing treating the filtration system associated with an air distribution system of an aircraft or like commercial passenger vehicle. At 200, one or more HEPA filters are provided in a duct or chamber of the air distribution system of an aircraft. At 202, at least one disinfecting UV array is provided in close proximity to at least one exposed surface of the at least one HEPA filter positioned in a duct or chamber of the air distribution system of the aircraft. Operation of the air distribution system of the aircraft produces the air stream is passed through the duct or chamber of the air distribution system of the aircraft at 204. Upon activation of the air distribution system to cause the air stream, the at least one disinfecting UV array irradiates the air stream adjacent the at least one HEPA filter and irradiates the exposed surface of the at least one HEPA filter, or other particulate filter at 206. Such an air stream may be generated by recirculating air from an interior cabin air space, or by directing a source of outside air through one or more ducts, or by a combination thereof for example.

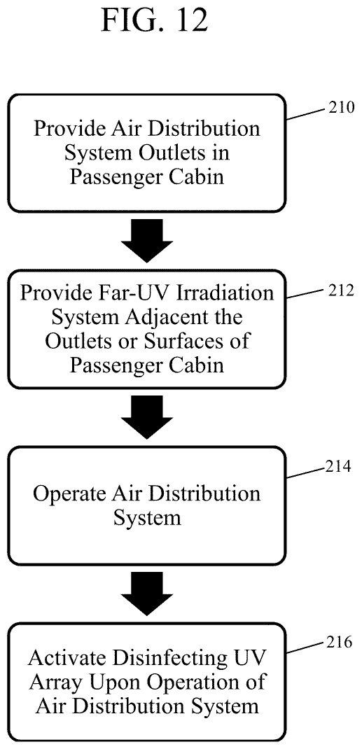

FIG. 12 schematically represents a series of steps involved in another method for treating the air circulated by an air distribution system of an aircraft or like commercial passenger vehicle and/or surfaces in the passenger seating areas of commercial passenger vehicles. At 210, the air distribution system of an aircraft includes blower outlets in the passenger compartment. At 212, at least one disinfecting far-UV array is provided in close proximity to the blower outlets in the passenger compartment or at least exposed surface of the passenger compartment of the aircraft. Operation of the air distribution system of the aircraft produces the air stream is passed through the blower outlets in the passenger compartment of the aircraft at 214. Upon activation of the air distribution system to cause the air stream, the at least one disinfecting far-UV array irradiates the air stream adjacent the blower outlets in the passenger compartment and/or irradiates at least one exposed surface of the passenger compartment.

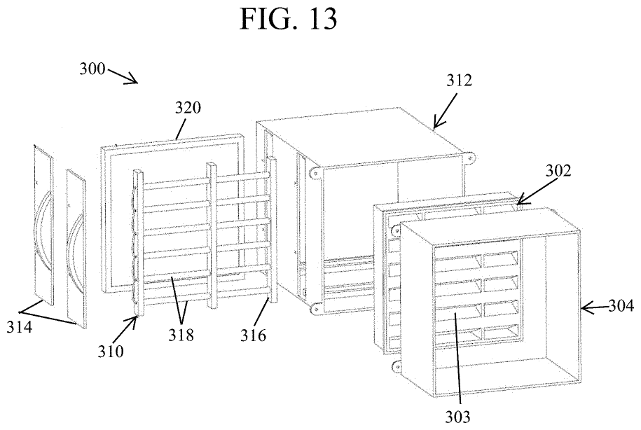

In another example with reference to FIG. 13, the UV sanitation assembly 300 is mounted and located in close proximity to the outer surface of the HEPA filter 302 mounted in the duct work 304 of an aircraft or like commercial passenger vehicle. In this example, the UVC emitters are instead an array of mercury lamps 310, mounted on close proximity to the exit side of the HEPA filter 302 in the flow of air to and from the passenger cabin. The use of an array of mercury lamps avoids the current low efficiency of UVC LED emitters. At this point, power conversion efficiency for UVC LED emitters is typically less than 5%. By comparison, UVC mercury lamps in the anti-germicidal wavelength band have a conversion efficiency of approximately 30% or greater, with maximum radiation levels approximately 10 times greater than the highest output UVC LED emitters currently available. The use of higher power radiation, and the location of the array 310 allow for enhanced germicidal effects to be achieved more quickly.

In this example, the positioning of the UV array 310 on the exhaust side of the HEPA filter 302 may be achieved by installation of the array 310 inside of an extension duct 312 attached to the existing ductwork 304. The extension duct 312 may be installed between the existing HEPA filter 302 and air system ductwork 304. The existing HEPA filter 302 may be mounted to the front of this extension ductwork 312 in the same manner that it attaches to the existing ductwork 304. The extension ductwork will be fabricated from or include an aluminum layer, which is highly reflective to UVC wavelengths to enhance distribution of the UVC radiation onto the HEPA filter 302. The extension ductwork 312 may have removable side access panels 314 so that the UVC lamp array 310 and/or HEPA filter 302 can be removed and serviced as necessary. In this example, the UVC mercury lamp array 310 is constructed on a sub-frame 316 supporting the UVC lamps 318, which slips into slots within the extension ductwork 312 so that the array 310 can be easily inserted or removed.

In this example, the positioning of the array 310 immediately downstream of the HEPA filter 302, allows the high power UVC mercury lamps 318 to be arrayed parallel with the surfaces of the HEPA filter 302 in a manner to best irradiate the pleats 303 on the exhaust side of the filter 302, as well as the air volume passing through the HEPA filter 302. If desired, a baffle arrangement may be used to slow the air volume an amount for treatment. The radiation from the UVC lamp array 310 will also serve to prevent any biofilm accumulation in the ductwork immediately following the HEPA filter.

To avoid exposure to harmful UVC radiation, disconnect switches, proximity sensing devices or other suitable arrangement may be employed on both the HEPA filter mounting surface and the side access panels 314 to the extension ductwork 312 so that removal of either will result in the deactivation of the UVC lamp array 310. The UVC mercury lamp array 310 may be powered by a ballast power supply (not shown) which is mounted externally to the ductwork. The ballast power supply may incorporate a timing and indicator circuit which monitors how long the UVC lamps have been in operation and signals when UVC lamp array 310 replacement is recommended based on known decay rate information for the UVC lamps being employed. In addition, a UVC power sensor may also be employed within the ductwork assembly to monitor both the operation and power output of the UVC lamp array 310. The ballast power supply may employ fault sensing circuits that will shut down the UVC array 310 in the event of UVC lamp malfunction.

The use of a high-power UVC sources 318, such as mercury lamps in this example or higher power LEDs in prior examples, also enables the use of a secondary UVC related purification system using Photo Catalytic Oxidation, or PCO. In this example, a PCO purification system 320 makes use of a photocatalytic agent, such as titanium dioxide (TiO2), which upon irradiation with high-power UV radiation, produces unstable hydroxyl radicals by means of interaction with water molecules in the air. The hydroxyl radicals in turn, readily react with any organic molecules in the air stream, yielding harmless carbon dioxide and water as byproducts. In this example, the PCO system 320 may be a filter or chamber coated with a reactive photocatalytic agent, adjacent to the UVC mercury lamp array 310. As the ambient air circulates through the PCO system 320, the reaction with any microbes by free hydroxy radicals and super-oxide ions created by UV light and titanium dioxide for example, results in breaking their cellular structure apart and destroying both the intracellular mass and DNA/HNA chromosomes. Thus, as the PCO system 320 also has effectiveness at eradicating airborne microbial pathogens, this enhances the overall effectiveness of the UVC/HEPA filter configuration for commercial passenger vehicles. In an example, the UV array 310 may also include UV radiation sources of a wavelength or range of wavelengths to optimize the photocatalytic reaction with the photocatalytic material in PCO system 320, such as around 365 nm for titanium dioxide, for example. Such sources could be UVA LEDs with this specific wavelength or range of wavelengths for use with the PCO system 320. With higher power UVC LED sources, there could also be provided a 275 nm UVC and/or 222 nm far UV LED array to irradiate the HEPA filter and air stream, with a supplementary 365 nm UVA LED array to irradiate the PCO filter using titanium dioxide. A further advantage of a PCO system 320 in association with the HEPA filter 302 in the commercial passenger vehicle environment is that whereas HEPA filtration is completely ineffective at reducing noxious and odorous gases in the airstream, PCO technology is highly effective at reducing these pollutants. With regard to air purification within a passenger aircraft cabin for example, VOC's (Volatile Organic Compounds) from sources such as jet exhaust, have become and remain an issue of concern. The PCO system 320 is effective at reducing VOC content or other noxious and odorous gases in the airstream. In this example, an optional PCO filter panel 320 would be installed downstream and in proximity to the UVC mercury lamp array 310 as illustrated, to allow the UVC radiation to catalyze the photocatalytic material.

Although certain examples of the invention have been described primarily with respect to supplying treated air to an interior space and surfaces of the interior space of an aircraft, the examples are not limiting and modifications may be made without departing from the spirit and scope of the invention as set forth in the following claims.

What is claimed is:

1. A sanitizing system for a commercial passenger vehicle comprising:
at least one air distribution system with at least one filter positioned in the air stream of the at least one air distribution system,
the at least one filter having an intake side and an exhaust side, and comprising filter media to remove contaminants,
at least one irradiation system positioned in proximity to at least one of the intake side or exhaust side of the at least one filter,
the at least one irradiation system including a plurality UV radiation sources in an array configured to irradiate exposed surfaces of the filter media with UV radiation of a predetermined wavelengths or range of wavelengths, and at a predetermined energy level; and
at least one support system for the array of UV radiation sources to position the array in predetermined relationship to the surfaces of the filter media in the at least one filter.

2. The sanitizing system of claim 1, wherein said filter media includes surface discontinuities to increase surface area, and the at least one support system positions UV radiation sources to irradiate all of the surface areas exposed by the surface discontinuities.

3. The sanitizing system of claim 2, wherein said surface discontinuities are pleats and the support system positions UV radiation sources in association with the pleats.

4. The sanitizing system of claim 1, wherein the system is configured to be positioned in the filter housing of the at least one air distribution system or adjacent the filter housing of the at least one air distribution system in association with an extension duct.

5. The sanitizing system of claim 1, wherein the plurality UV radiation sources are selected from the group consisting of UV LED's, UV mercury lamps, UV excimer lamps or combinations thereof.

6. The sanitizing system of claim 1, wherein the filter media is formed in a substantially cylindrical form and the array is configured to irradiate the cylindrical surfaces of the at least one filter.

7. The sanitizing system of claim 6, wherein the array is positioned on the exhaust side of the at least one filter, interior to the cylindrical form or on the intake side of the at least one filter, exterior to the cylindrical form.

8. The sanitizing system of claim 1, further comprising a photo catalytic oxidation (PCO) system positioned in proximity to the at least one irradiation system.

9. The sanitizing system of claim 1, further comprising a reflector associated with at least one UV radiation source positioned to reflect UV radiation toward the at least one filter.

10. The sanitizing system of claim 1, wherein the predetermined wavelengths of the plurality of UV radiation sources is between 200 and 280 nm.

11. The sanitizing system of claim 1, wherein the array includes a plurality of UV LED emitters provided on PCB assemblies on a support, the support configured to correspond to the shape and configuration of the at least one filter.

12. The sanitizing system of claim 1, wherein the system includes at least one structure to cause the speed of the circulated air to be slowed for treatment by the at least one irradiation system.

13. The sanitizing system of claim 1, further comprising an air motion sensor and associated system to turn the array on in the event that the air distribution system is activated and off in the event that the air distribution system is deactivated.

14. The sanitizing system of claim 1, wherein at least one sanitizing UV array is provided in a position adjacent to at least one outlet of an air distribution system of the commercial passenger vehicle that recirculates air to the passenger cabin of the commercial passenger vehicle or at least one surface in the cabin to treat air distributed to the passenger cabin of the commercial passenger vehicle from at least one outlet of an air distribution system of the commercial passenger vehicle.

15. The sanitizing system of claim 1, wherein the at least one irradiation system is provided with the at least one filter to provide a drop-in replacement for an existing filter of the commercial passenger vehicle.

16. The sanitizing system of claim 1, wherein the at least one irradiation system is powered using a power supply of the commercial passenger vehicle.

17. The sanitizing system of claim 1, wherein the at least one irradiation system is operated at a precise operating current level over temperature.

18. The sanitizing system of claim 1, wherein the at least one irradiation system is mounted on a framework and does not substantially impede air flow through the at least one filter.

19. The sanitizing system of claim 1, wherein the at least one irradiation system further includes at least one pre-filter.

20. A method for sanitizing in a commercial passenger vehicle comprising:
providing at least one filter in an air distribution system of the commercial passenger vehicle;
positioning at least one sanitizing UV array in close proximity to at least one exposed surface of the at least one filter in the air distribution system;
causing the flow of air through the air distribution system and at least one filter and activating the at least one sanitizing UV array to irradiate the air stream adjacent the at least one filter and irradiate the exposed surfaces of the at least one filter, and distributing air treated by the at least one sanitizing UV array and at least one filter to a passenger cabin of the commercial passenger vehicle.
